(19) United States
(12) Patent Application Publication
(10) Pub. No.: US 2023/0381391 A1
(43) Pub. Date: Nov. 30, 2023

(54) The Use of a Hemocompatible Porous Polymer Bread Sorbent for Removal of Pamps and Damps

(71) Applicant: CytoSorbents, Inc., Princeton, NJ (US)

(72) Inventors: Maryann GRUDA, Princeton, NJ (US); Pamela O'SULLIVAN, Princeton, NJ (US); Tamaz GULIASHVILI, Princeton, NJ (US); Andrew SCHEIRER, Bethlehem, PA (US); Thomas GOLOBISH, Bethlehem, PA (US); Vincent CAPPONI, Monmouth Junction, NJ (US); Philip CHAN, Cherry Hill, NJ (US)

(21) Appl. No.: 18/110,648

(22) Filed: Feb. 16, 2023

Related U.S. Application Data
(63) Continuation of application No. 16/082,376, filed on Sep. 5, 2018.
(60) Provisional application No. 62/305,382, filed on Mar. 8, 2016.

(51) Int. Cl. A61M 1/34; A61M 1/36; B01J 20/26; B01J 20/28

(52) U.S. Cl. CPC A61M 1/3486; A61M 1/3679; B01J 20/267; B01J 20/28083; B01J 20/28085; B01J 20/28019; B01J 20/2808; B01J 20/261

(57) ABSTRACT

The invention concerns biocompatible polymer systems comprising at least one polymer sorbent with a plurality of pores, said polymer designed to adsorb pathogen-associated molecular pattern molecules and damage-associated molecular pattern molecules. Also disclosed herein are methods for reducing contamination in a biological substance, or treating contamination in a subject, by one or more pathogen-associated molecular pattern molecules and damage-associated molecular pattern molecules, by contacting the biological substance with an effective amount of sorbent capable of sorbing the toxin.

### CROSS REFERENCE TO RELATED APPLICATIONS

The present application claims benefit of U.S. Patent Application No. 62/305,382 filed on Mar. 8, 2016, the disclosure of which is incorporated herein in its entirety.

### GOVERNMENT RIGHTS

The subject matter disclosed herein was made with government support under contract number N66001-12-C-4199, awarded by The Defense Advanced Research Projects Agency (DARPA). The government may have certain rights in the herein disclosed subject matter.

### TECHNICAL FIELD

The disclosed inventions are in the field of porous polymeric sorbents. The disclosed inventions are also in the field of broadly reducing pathogen-associated molecular pattern molecules and damage-associated molecular pattern molecules in blood, blood products, and other physiologic fluids Additionally, the disclosed inventions are in the field of broadly removing pathogen-associated molecular pattern molecules and damage-associated molecular pattern molecules by static adsorption, perfusion, or hemoperfusion.

### BACKGROUND

Prolonged and upregulated inflammatory responses may lead to sepsis or systemic inflammatory response syndrome (SIRS), both of which can progress to potentially fatal septic shock and multiple organ dysfunction syndrome (MODS). Sepsis and septic shock result from a life-threatening systemic inflammatory response syndrome (SIRS) to invading pathogens or direct tissue insults. Sepsis is a highly heterogeneous disease with severity and progression dependent upon a myriad of interacting factors, including: the microbial insult, which may be of bacterial (gram-positive and gram-negative), viral, fungal or parasitic origin; the pathogen load, toxin production, virulence; host factors such as age, genetic composition, and comorbidities; the site of infection as well as the elapsed time since the initial infection. This complexity creates a highly dynamic and unstable situation that has confounded therapeutic efforts targeted to specific factors.

Examples of pathogens commonly associated with the development of sepsis are Staphylococcus species including Staphylococcus aureus (S. aureus), Streptococcus species such as Streptococcus pneumonia, Streptococcus pyogenes (S. pyogenes), Klebsiella species, Escherichia coli (E. coli), Pseudomonas species such as Pseudomonas aeruginosa (P. aeruginosa), Listeria species, several fungal species (e.g. Aspergillus, Fusarium and Candida subspecies, as well as viruses (such as Dengue and influenza viruses) and parasites. These pathogens release or cause the release of a daunting array of virulence factors that modulate the immune response and influence the severity of the disease. The host response to pathogenic insults involves multiple sequential and concurrent processes that produce both exaggerated inflammation and immune suppression. Pathogen-associated molecular pattern molecules (PAMPs), such as lipopolysaccharides, lipopeptides, lipoteichoic acid, peptidoglycans, nucleic acids such as double-stranded RNA, toxins and flagellins, trigger an immune response in the host (e.g. the innate immune system) to fight the infection, leading to the production of high levels of inflammatory and anti-inflammatory mediators, such as cytokines. PAMPs and high cytokine levels, as well as direct tissue injury (trauma, burns, etc.), can damage tissue, causing the extracellular release of damage-associated molecular pattern (DAMPs) molecules into the bloodstream. DAMPS are a broad class of endogenous molecules, which like PAMPs, trigger the immune response through pattern recognition receptors (PRRs) such as Toll-like receptors (TLRs).

DAMPs have also been associated with countless syndromes and diseases. These include complications from trauma, burns, traumatic brain injury and invasive surgery, and also organ-specific illnesses like liver disease, kidney dialysis complications, and autoimmune diseases. DAMPs are host molecules that can initiate and perpetuate noninfectious SIRS and exacerbate infectious SIRS. DAMPs are a diverse family of molecules that are intracellular in physiological conditions and many are nuclear or cytosolic proteins. DAMPS can be divided into two groups: (1) molecules that perform noninflammatory functions in living cells (such as HMGB1) and acquire immunomodulatory properties when released, secreted, modified, or exposed on the cell surface during cellular stress, damage, or injury, or (2) alarmins, i.e., molecules that possess cytokine-like functions (such as (3-Defensins and Cathelicidin), which can be stored in cells and released upon cell lysis, whereupon they contribute to the inflammatory response. When released outside the cell or exposed on the surface of the cell following tissue injury, they move from a reducing to an oxidizing milieu, which affects their activity. Also, following necrosis, mitochondrial and nuclear DNA fragments are released outside the cell becoming DAMPs.

DAMPs, such as HMGB-1, heat-shock and 5100 proteins are normally found inside cells and are released by tissue damage. DAMPs act as endogenous danger signals to promote and exacerbate the inflammatory response. HMGB-1 is a non-histone nuclear protein that is released under stress conditions. Extracellular HMGB-1 is an indicator of tissue necrosis and has been associated with an increased risk of sepsis and multiple organ dysfunction syndrome (MODS). 5100 A8 (granulin A, MRP8) and A9 (granulin B\, MRP14) homo and heterodimers bind to and signal directly via the TLR4/lipopolysaccharide receptor complex where they become danger signals that activate immune cells and vascular endothelium. Procalcitonin is a marker of severe sepsis caused by bacteria and its release into circulation is indicative of the degree of sepsis. Serum amyloid A (SAA), an acute-phase protein, is produced predominantly by hepatocytes in response to injury, infection, and inflammation. During acute inflammation, serum SAA levels may rise by 1000-fold. SAA is chemotactic for neutrophils and induces the production of proinflammatory cytokines. Heat shock proteins (HSP) are a family of proteins that are produced by cells in response to exposure to stressful conditions and are named according to their molecular weight (10, 20-30, 40, 60, 70, 90). The small 8-kilodalton protein ubiquitin, which marks proteins for degradation, also has features of a heat shock protein. Hepatoma-derived growth factor (HDGF), despite its name, is a protein expressed by neurons. HDGF can be released actively by neurons via a nonclassical pathway and passively by necrotic cells. Other factors, such as complement factors 3 and 5, are activated as part of the host defense against pathogens but can also contribute to the adverse outcomes in sepsis. Excessive, persistent circulating levels of cytokines and DAMPs contribute to organ injury and identify those patients who have the highest risk of multiple organ dysfunction (MODs) and death in community acquired pneumonia and sepsis.

Staphylococcus aureus, the leading cause of gram positive bacteremia, is associated with higher morbidity and mortality largely due to the increase in methicillin-resistant S. aureus (MRSA). S. aureus is effective in invading the bloodstream and evading the host immunological response due to a variety of PAMPs, such as Panton-Valentine leukocidin (PVL), a cytolysin produced by many S. aureus clinical isolates that functions as a key virulence factor by forming pores in cell membranes. Streptococcus pneumoniae and Listeria monocytogenes are also gram-positive bacteria that produce the pore forming toxins pneumolysin, streptolysin and listeriolysin that facilitate infection by damaging host cells and interfering with the host immune response.

Superantigens are a class of antigens that cause non-specific activation of T-cells resulting in polyclonal T cell activation and massive cytokine release. Superantigens are produced by some pathogenic viruses and bacteria most likely as a defense mechanism against the immune system. Staphylococcal and Streptococcal superantigens form a large protein family having all evolved from a single primordial superantigen. In particular, Streptococcus pyrogenic exotoxins (SPEs) A, C, G-M, S. aureus TSST-1 toxin, and Y. pseudotuberculosis YPM-a and YPM-b are superantigens. The nucleocapsid (N) protein of rabies virus is reported to be a superantigen in humans, stimulating Vb8T lymphocytes.

Staphylococcal A and B (ETA and ETB), that produces Staphylococcal Scalded Skin Syndrome (SSSS) are serine proteases that belong to the class of exfoliative toxins. Hypotension and possible organ failure can be found in severe cases of SSSS where there are extensive areas of denuded skin with significant fluid loss or with a secondary infecting organism.

Streptococcus pyogenes is a group A streptococcus (GAS) that utilizes several virulence factors, Spe A to G, to establish infection. Of these, the streptococcus pyrogenic exotoxin B (SpeB), cleaves or degrades host immunoglobulin and complement components to evade the immune response by inhibiting phagocytic activity (Kuo 2008).

Bacterial flagellins are bacterial structural protein that elicits immune response via toll-like receptor 5, a PRR. Flagellins are extraordinarily potent proinflammatory stimuli in the lungs during sepsis. Flagellins induce a local release of proinflammatory cytokines, the accumulation of inflammatory cells, and the development of pulmonary hyperpermeability. Numerous forms of flagellin are made by bacteria with E. coli produced flagellin ranging in size from 37 to 69 kDa.

Over 20 Aspergillus species are known to cause human disease. Invasive aspergillosis is a devastating infectious disease that mainly affects critically ill and immunocompromised patients. Aspergillus fumigatus is the most prevalent and is largely responsible for the increased incidence of invasive aspergillosis (IA) in the immunocompromised patient population. IA is a devastating illness, with mortality rates in some patient groups reaching as high as 90%. Aspergillus species produce a variety of mycotoxins, such as gliotoxin, that contribute to pathogenicity by host immunosuppression, and aflatoxin that can cause acute hepatic injury and liver failure. Fusarium species cause a broad spectrum of infections in humans, including superficial, locally invasive, and disseminated infections. Fusarium species possess several virulence factors, including mycotoxins, such as T-2 toxin, a trichothecene mycotoxin, which suppresses humoral and cellular immunity and may also cause tissue breakdown.

### SUMMARY

In some aspects, the invention concerns a biocompatible polymer system comprising at least one polymer; the polymer system capable of adsorbing (i) pathogen-associated molecular pattern molecules and (ii) damage-associated molecular pattern molecules having a molecular weight of from less than about 0.5 kDa to about 1,000 kDa (or about 1 kDa to about 1,000 kDa or about 0.1 kDa to about 1,000 kDa in some embodiments). Some preferred polymers are hemocompatible. Certain preferred polymer systems have geometry of a spherical bead.

Some polymer systems have a polymer pore structure that has a total volume of pore sizes in the range of from 50 Å to 40,000 Å greater than 0.5 cc/g and less than 5.0 cc/g dry polymer.

In some embodiments, the toxins adsorbed comprise one or more of PAMPs and DAMPS comprised of one or more of flagellins, lipopeptides, formyl peptides, mycotoxins, exotoxins, endotoxins, lipoteichoic acid, cytolysins, superantigens, proteases, lipases, amylases, enzymes, peptides including bradykinin, activated complement, soluble receptors, soluble CD40 ligand, bioactive lipids, oxidized lipids, cellular DNA, mitochondrial DNA, pathogen or host derived RNA, cell-free hemoglobin, cell-free myoglobin, growth factors, peptidoglycans, glycoproteins, released intracellular components, cell wall or viral envelope components, Polyinosinic:polycytidylic acid (poly I:C), prions, toxins, bacterial and viral toxins, drugs, vasoactive substances, and foreign antigens.

The polymers can be made by any means known in the art to produce a suitable porous polymer. In some embodiments, the polymer is made using suspension polymerization. Some polymers comprise a hypercrosslinked polymer. Certain spherical beads have a biocompatible hydrogel coating.

Certain polymers are formed and subsequently modified to be biocompatible. Some modifications comprise forming a biocompatible surface coating or layer.

Other aspects include methods of perfusion comprising passing a physiologic fluid once, or by way of a suitable extracorporeal circuit, through a device comprising the biocompatible polymer system one or more times described herein.

Yet another aspect concerns devices for removing (i) pathogen-associated molecular pattern molecules and (ii) damage-associated molecular pattern molecules from less than 0.5 kDa to 1,000 kDa from physiologic fluid comprising the biocompatible polymer system described herein.

### BRIEF DESCRIPTION OF THE DRAWINGS

The accompanying drawings, which are included to provide a further understanding of the disclosure, are incorporated in and constitute a part of this specification, illustrate aspects of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIGS. 1 and 2 present DAMPS and PAMPs removal data from an in vitro dynamic model, using whole blood, expressed as percentage remaining compared to the pre-circulation concentrations for modified polymer CY15065.

FIGS. 3 and 4 presents DAMPs and PAMPs removal data from an in vitro dynamic model, using whole blood, expressed as percentage remaining compared to the pre-circulation concentrations for polymer CY15077.

### DETAILED DESCRIPTION OF ILLUSTRATIVE EMBODIMENTS

As required, detailed embodiments of the present invention are disclosed herein; it is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the invention to be better understood. However, they are given merely by way of guidance and do not imply any limitation.

The present invention may be understood more readily by reference to the following detailed description taken in connection with the accompanying figures and examples, which form a part of this disclosure. It is to be understood that this invention is not limited to the specific materials, devices, methods, applications, conditions or parameters described and/or shown herein, and that the terminology used herein is for the purpose of describing particular embodiments by way of example only and is not intended to be limiting of the claimed invention. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention, which are, for clarity, described herein in the context of separate embodiments, may also be provided in combination in a single embodiment. Conversely, various features of the invention that are, for brevity, described in the context of a single embodiment, may also be provided separately or in any subcombination. Further reference to values stated in ranges includes each and every value within that range.

The following definitions are intended to assist in understanding the present invention:

Pathogen-associated molecular pattern molecules (PAMPS) are molecules derived from microorganisms that are recognized by cells of the innate immune system. These molecules have small molecular motifs conserved within a class of microbes that are recognized by toll-like receptor and pattern recognition receptors that initiate and perpetuate a pathogen-induced inflammatory response.

Damage-associated molecular pattern molecules (DAMPS) are host biomolecules released by stressed cells that initiate and perpetuate inflammation in response to trauma, ischemia, and tissue damage either in the absence or presence of pathogenic infection.

The term “biocompatible” is defined to mean the sorbent is capable of coming in contact with physiologic fluids, living tissues, or organisms, without producing unacceptable clinical changes during the time that the sorbent is in contact with the physiologic fluids, living tissues, or organisms.

The term “hemocompatible” is defined as a condition whereby a biocompatible material when placed in contact with whole blood or blood plasma results in clinically acceptable physiologic changes.

As used herein, the term “sorbent” includes adsorbents and absorbents.

For purposes of this invention, the term “sorb” is defined as “taking up and binding by absorption and adsorption”.

For the purposes of this invention, the term “perfusion” is defined as passing a physiologic fluid, once through or by way of a suitable extracorporeal circuit, through a device containing the porous polymeric adsorbent to remove toxic molecules from the fluid.

The term “hemoperfusion” is a special case of perfusion where the physiologic fluid is blood.

The term “dispersant” or “dispersing agent” is defined as a substance that imparts a stabilizing effect upon a finely divided array of immiscible liquid droplets suspended in a fluidizing medium.

The term “macroreticular synthesis” is defined as a polymerization of monomers into polymer in the presence of an inert precipitant which forces the growing polymer molecules out of the monomer liquid at a certain molecular size dictated by the phase equilibria to give solid nanosized microgel particles of spherical or almost spherical symmetry packed together to give a bead with physical pores of an open cell structure [U.S. Pat. No. 4,297,220, Meitzner and Oline, Oct. 27, 1981; R. L. Albright, Reactive Polymers, 4, 155-174(1986)].

The term “hypercrosslinked” describes a polymer in which the single repeating unit has a connectivity of more than two. Hypercrosslinked polymers are prepared by crosslinking swollen, or dissolved, polymer chains with a large number of rigid bridging spacers, rather than copolymerization of monomers. Crosslinking agents may include bis(chloromethyl) derivatives of aromatic hydrocarbons, methylal, monochlorodimethyl ether, and other bifunctional compounds that react with the polymer in the presence of Friedel-Crafts catalysts [Tsyurupa, M. P., Z. K. Blinnikova, N. A. Proskurina, A. V. Pastukhov, L. A. Pavlova, and V. A. Davankov. “Hypercrosslinked Polystyrene: The First Nanoporous Polymeric Material.” Nanotechnologies in Russia 4 (2009): 665-75.]

Some preferred polymers comprise residues from one or more monomers, or containing monomers, or mixtures thereof, selected from acrylonitrile, allyl glycidyl ether, butyl acrylate, butyl methacrylate, cetyl acrylate, cetyl methacrylate, 3,4-dihydroxy-1-butene, dipentaerythritol diacrylate, dipentaerythritol dimethacrylate, dipentaerythritol tetraacrylate, dipentaerythritol tetramethacrylate, dipentaerythritol triacrylate, dipentaerythritol trimethacrylate, divinylbenzene, divinylformamide, divinylnaphthalene, divinylsulfone, 3,4-epoxy-1-butene, 1,2-epoxy-9-decene, 1,2-epoxy-5-hexene, ethyl acrylate, ethyl methacrylate, ethylstyrene, ethylvinylbezene, glycidyl methacrylate, methyl acrylate, methyl methacrylate, octyl acrylate, octyl methacrylate, pentaerythritol diacrylate, pentaerythritol dimethacrylate, pentaerythritol tetraacrylate, pentaerythritol tetramethacrylate, pentaerythritol triacrylate, pentaerythritol trimethacrylate, styrene, trimethylolpropane diacrylate, trimethylolpropane dimethacrylate, trimethylolpropane triacrylate, trimethylolpropane trimethacrylate, trivinylbenzene, trivinylcyclohexane, vinyl acetate, vinylbenzyl alcohol, 4-vinyl-1-cyclohexene 1,2-epoxide, vinylformamide, vinylnaphthalene, 2-vinyloxirane, and vinyltoluene.

Some embodiments of the invention use an organic solvent and/or polymeric porogen as the porogen or pore-former, and the resulting phase separation induced during polymerization yield porous polymers. Some preferred porogens are selected from, or mixtures comprised of any combination of, benzyl alcohol, cyclohexane, cyclohexanol, cyclohexanone, decane, dibutyl phthalate, di-2-ethylhexyl phthalate, di-2-ethylhexylphosphoric acid, ethylacetate, 2-ethyl-1-hexanoic acid, 2-ethyl-1-hexanol, n-heptane, n-hexane, isoamyl acetate, isoamyl alcohol, n-octane, pentanol, poly(propylene glycol), polystyrene, poly(styrene-co-methyl methacrylate), tetraline, toluene, tri-n-butylphosphate, 1,2,3-trichloropropane, 2,2,4-trimethylpentane, and xylene.

In yet another embodiment, the dispersing agent is selected from a group consisting of hydroxyethyl cellulose, hydroxypropyl cellulose, poly(diethylaminoethyl acrylate), poly(diethylaminoethyl methacrylate), poly(dimethylaminoethyl acrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(vinyl alcohol), salts of poly(acrylic acid), salts of poly(methacrylic acid) and mixtures thereof.

Preferred sorbents are biocompatible. In another further embodiment, the polymer is biocompatible. In yet another embodiment, the polymer is hemocompatible. In still a further embodiment, the biocompatible polymer is hemocompatible. In still a further embodiment, the geometry of the polymer is a spherical bead.

In another embodiment, the biocompatible polymer comprises poly(N-vinylpyrrolidone).

The coating/dispersant on the poly(styrene-co-divinylbenzene) resin will imbue the material with improved biocompatibility.

In still yet another embodiment, a group of cross-linkers consisting of dipentaerythritol diacrylates, dipentaerythritol dimethacrylates, dipentaerythritol tetraacrylates, dipentaerythritol tetramethacrylates, dipentaerythritol triacrylates, dipentaerythritol trimethacrylates, divinylbenzene, divinylformamide, divinylnaphthalene, divinylsulfone, pentaerythritol diacrylates, pentaerythritol dimethacrylates, pentaerythritol tetraacrylates, pentaerythritol tetramethacrylates, pentaerythritol triacrylates, pentaerythritol trimethacrylates, trimethylolpropane diacrylate, trimethylolpropane dimethacrylate, trimethylolpropane triacrylate, trimethylolpropane trimethacrylate, trivinylbenzene, trivinylcyclohexane and mixtures thereof can be used in formation of a hemocompatible hydrogel coating.

In some embodiments, the polymer is a polymer comprising at least one crosslinking agent and at least one dispersing agent. The dispersing agent may be biocompatible. The dispersing agents can be selected from chemicals, compounds or materials such as hydroxyethyl cellulose, hydroxypropyl cellulose, poly(diethylaminoethyl acrylate), poly(diethylaminoethyl methacrylate), poly(dimethylaminoethyl acrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(vinyl alcohol), salts of poly(acrylic acid), salts of poly(methacrylic acid) and mixtures thereof; the crosslinking agent selected from a group consisting of dipentaerythritol diacrylates, dipentaerythritol dimethacrylates, dipentaerythritol tetraacrylates, dipentaerythritol tetramethacrylates, dipentaerythritol triacrylates, dipentaerythritol trimethacrylates, divinylbenzene, divinylformamide, divinylnaphthalene, divinylsulfone, pentaerythritol diacrylates, pentaerythritol dimethacrylates, pentaerythritol tetraacrylates, pentaerythritol tetramethacrylates, pentaerythritol triacrylates, pentaerythritol trimethacrylates, trimethylolpropane diacrylate, trimethylolpropane dimethacrylate, trimethylolpropane triacrylate, trimethylolpropane trimethacrylate, trivinylbenzene, trivinylcyclohexane and mixtures thereof. Preferably, the polymer is developed simultaneously with the formation of the coating, wherein the dispersing agent is chemically bound or entangled on the surface of the polymer.

In still another embodiment, the biocompatible polymer coating is selected from a group consisting of poly(diethylaminoethyl methacrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(N-vinylpyrrolidone), poly(vinyl alcohol), salts of poly(acrylic acid), salts of poly(methacrylic acid) and mixtures thereof.

In still another embodiment, the biocompatible oligomer coating is selected from a group consisting of poly(diethylaminoethyl methacrylate), poly(dimethylaminoethyl methacrylate), poly(hydroxyethyl acrylate), poly(hydroxyethyl methacrylate), poly(hydroxypropyl acrylate), poly(hydroxypropyl methacrylate), poly(N-vinylpyrrolidone), poly(vinyl alcohol), salts of poly(acrylic acid), salts of poly(methacrylic acid) and mixtures thereof.

Some present biocompatible sorbent compositions are comprised of a plurality of pores. The biocompatible sorbents are designed to adsorb a broad range of toxins from less than kDa to 1,000 kDa. While not intending to be bound by theory, it is believed the sorbent acts by sequestering molecules of a predetermined molecular weight within the pores. The size of a molecule that can be sorbed by the polymer will increase as the pore size of the polymer increases. Conversely, as the pore size is increased beyond the optimum pore size for adsorption of a given molecule, adsorption of said protein may or will decrease.

In certain methods, the solid form is porous. Some solid forms are characterized as having a pore structure having a total volume of pore sizes in the range of from 50 Å to greater than 0.5 cc/g and less than 5.0 cc/g dry polymer.

In one embodiment, the sorbent has a pore structure wherein at least ⅓ of the pore volume in pores having diameters between 50 Å and 40,000 Å is in pores having diameters between 100 Å and 1,000 Å.

In another embodiment, the sorbent has a pore structure wherein at least ½ of the pore volume in pores having diameters between 50 Å and 40,000 Å is in pores having diameters between 1000 Å and 10,000 Å.

In still another embodiment, the sorbent has a pore structure wherein at least 1/3 of the pore volume in pores having diameters between 50 Å and 40,000 Å is in pores having diameters between 10,000 Å and 40,000 Å.

In certain embodiments, the polymers can be made in bead form having a diameter in the range of 0.1 micrometers to 2 centimeters. Certain polymers are in the form of powder, beads or other regular or irregularly shaped particulates.

In some embodiments, the plurality of solid forms comprises particles having a diameter in the range for 0.1 micrometers to 2 centimeters.

In some methods, the undesirable molecules include PAMPs and DAMPS comprised of one or more of flagellins, lipopeptides, formyl peptides, mycotoxins, exotoxins, cytolysins, superantigens, proteases, lipases, amylases, enzymes, peptides including bradykinin, activated complement, soluble receptors, soluble CD40 ligand, bioactive lipids, oxidized lipids, cell-free hemoglobin, cell-free myoglobin, growth factors, glycoproteins, prions, toxins, bacterial and viral toxins, drugs, vasoactive substances, foreign antigens, and antibodies.

In one embodiment, the polymers of this invention are made by suspension polymerization in a formulated aqueous phase with free radical initiation in the presence of aqueous phase dispersants that are selected to provide a biocompatible and a hemocompatible exterior surface to the formed polymer beads. In some embodiments, the beads are made porous by the macroreticular synthesis with an appropriately selected porogen (pore forming agent) and an appropriate time-temperature profile for the polymerization in order to develop the proper pore structure.

In another embodiment, polymers made by suspension polymerization can be made biocompatible and hemocompatible by further grafting of biocompatible and hemocompatible monomers or low molecular weight oligomers. It has been shown that the radical polymerization procedure does not consume all the vinyl groups of DVB introduced into copolymerization. On average, about 30% of DVB species fail to serve as crosslinking bridges and remain involved in the network by only one of two vinyl groups. The presence of a relatively high amount of pendant vinyl groups is therefore a characteristic feature of the adsorbents. It can be expected that these pendant vinyl groups are preferably exposed to the surface of the polymer beads and their macropores, if present, should be readily available to chemical modification. The chemical modification of the surface of DVB-copolymers relies on chemical reactions of the surface-exposed pendant vinyl groups and aims at converting these groups into more hydrophilic functional groups. This conversion via free radical grafting of monomers and/or cross-linkers or low molecular weight oligomers provides the initial hydrophobic adsorbing material with the property of hemocompatibility.

In yet another embodiment, the radical polymerization initiator is initially added to the dispersed organic phase, not the aqueous dispersion medium as is typical in suspension polymerization. During polymerization, many growing polymer chains with their chain-end radicals show up at the phase interface and can initiate the polymerization in the dispersion medium. Moreover, the radical initiator, like benzoyl peroxide, generates radicals relatively slowly. This initiator is only partially consumed during the formation of beads even after several hours of polymerization. This initiator easily moves toward the surface of the bead and activates the surface exposed pendant vinyl groups of the divinylbenzene moiety of the bead, thus initiating the graft polymerization of other monomers added after the reaction has proceeded for a period of time. Therefore, free-radical grafting can occur during the transformation of the monomer droplets into polymer beads thereby incorporating monomers and/or cross-linkers or low molecular weight oligomers that impart biocompatibility or hemocompatibility as a surface coating.

The hemoperfusion and perfusion devices consist of a packed bead bed of the polymer beads in a flow-through container fitted with either a retainer screen at both the exit end and the entrance end to maintain the bead bed inside the container, or with a subsequent retainer screen to collect the beads after mixing. The hemoperfusion and perfusion operations are performed by passing the whole blood, blood plasma or physiologic fluid through the packed bead bed. During the perfusion through the bead bed, the toxic molecules are retained by sorption, torturous path, and/or pore capture, while the remainder of the fluid and intact cell components pass through essentially unchanged in concentration.

In some other embodiments, an in-line filter is comprised of a packed bead bed of the polymer beads in a flow-through container, fitted with a retainer screen at both the exit end and the entrance end to maintain the bead bed inside the container. Biological fluids are passed from a storage bag once-through the packed bead bed via gravity, during which the toxic molecules are retained by sorption, torturous path, and/or pore capture, while the remainder of the fluid and intact cell components pass through essentially unchanged in concentration.

Certain polymers useful in the invention (as is or after further modification) are macroporous polymers prepared from the polymerizable monomers of styrene, divinylbenzene, ethylvinylbenzene, and the acrylate and methacrylate monomers such as those listed below by manufacturer. Rohm and Haas Company, (now part of Dow Chemical Company): macroporous polymeric sorbents such as Amberlite™ XAD-1, Amberlite™ XAD-2, Amberlite™ XAD-4, Amberlite™ XAD-7, Amberlite™ XAD-7HP, Amberlite™ XAD-8, Amberlite™ XAD-16, Amberlite™ XAD-16 HP, Amberlite™ XAD-18, Amberlite™ XAD-200, Amberlite™ XAD-1180, Amberlite™ XAD-2000, Amberlite™ XAD-2005, Amberlite™ XAD-2010, Amberlite™ XAD-761, and Amberlite™ XE-305, and chromatographic grade sorbents such as Amberchrom™ CG 71,s,m,c, Amberchrom™ CG 161,s,m,c, Amberchrom™ CG 300,s,m,c, and Amberchrom™ CG 1000,s,m,c. Dow Chemical Company: Dowex™ Optipore™ L-493, Dowex™ Optipore™ V-493, Dowex™ Optipore™ V-502, Dowex™ Optipore™ L-285, Dowex™ Optipore™ L-323, and Dowex™ Optipore™ V-503. Lanxess (formerly Bayer and Sybron): Lewatit™ VPOC 1064 MD PH, Lewatit™ VPOC 1163, Lewatit™ OC EP 63, Lewatit™ S 6328 A, Lewatit™ OC 1066, and Lewatit™ 60/150 MIBK. Mitsubishi Chemical Corporation: Diaion™ HP 10, Diaion™ HP 20, Diaion™ HP 21, Diaion™ HP 30, Diaion™ HP Diaion™ HP 50, Diaion™ SP70, Diaion™ SP 205, Diaion™ SP 206, Diaion™ SP 207, Diaion™ SP 700, Diaion™ SP 800, Diaion™ SP 825, Diaion™ SP 850, Diaion™ SP 875, Diaion™ HP 1MG, Diaion™ HP 2MG, Diaion™ CHP 55A, Diaion™ CHP 55Y, Diaion™ CHP Diaion™ CHP 20Y, Diaion™ CHP 2MGY, Diaion™ CHP 20P, Diaion™ HP 20SS, Diaion™ SP 20SS, Diaion™ SP 207SS. Purolite Company: Purosorb™ AP 250 and Purosorb™ AP 400, and Kaneka Corp. Lixelle and CTR beads.and BioSKY™ MG Blood Perfusion Column and polymers within, BioSKY™ DX Bilirubin Perfusion Column and polymers within, Jafron Columns/Cartridges and polymers within such as BS330, DNA230, HA130, HA230, HA280, HA330, and HA330-II.

Various DAMPs and PAMPs may be adsorbed by the composition of the instant disclosure Some of these proteins and their molecular weights are shown in the table below.

| | Molecular |
|---|---|
| Protein | Weight (Da) |
| Gliotoxin | 326 |
| T-2 toxin | 466 |
| PAF (Platelet Activating Factor) | 524 |
| Bilirubin | 549 |
| Heme b | 616 |
| Lipopeptides | 607-1,536 |
| Formyl peptides | 437-3,600 |
| cathelicidin | 4,500 |
| β-Defensin | 7,000 |
| Ubiquitin | 8000 |
| Complement C5a | 8,200 |
| Complement C3a | 9,089 |
| Heat shock protein 10 | 10,000 |
| S100B (monomer) | 10,000 |
| Histone H4 | 11,000 |
| Procalcitonin | 13,000 |
| Serum amyloid A | 13,000 |
| Phospholipase A2, secretory PLA2 | 14,000 |
| PLA2G2A | 16,083 |
| S100 proteins A8 (dimer) | 20,000 |
| Trypsin | 23,300 |
| Staph Enterotoxin B | 24,500 |
| HMGB1 | 24,894 |
| Chymotrypsin | 25,000 |
| Elastase (neutrophil) | 25,000 |
| Staph ETA | 26,900 |
| HDGF | 27,000 |
| PF4 | 27,100 |
| Staph ETB | 27,300 |
| PCNA, proliferating cell | 29,000 |
| nuclear antigen | |
| Panton-Valentine leukocidin | 34,000 |
| Arginse I | 35,000 |
| Carboxypeptidase A | 35,000 |
| Thrombin | 36,700 |
| Flagellin H4 | 37,000 |
| Flagellin H17 | 39,000 |
| Heat shock protein 40 | 40,000 |
| Strep pyrogenic exotoxin B | 42,000 |
| alpha-1 antitrypsin | 44,324 |
| Soluble CD14 | 49,000 |
| Rabies nucleocapsid N protein | 50,600 |
| Soluble TNF-α receptor | 55,000 |
| Activated Protein C | 56,200 |
| Amylase | 57,000 |
| Hemopexin | 57,000 |
| Lysteriolysin | 58,000 |
| Heat shock protein 60 | 60,000 |
| Streptolysin O | 60,500 |
| Diptheria toxoid | 62,000 |
| Hemoglobin, oxy | 64,000 |
| Pseudomonas Exotoxin A | 66,000 |
| Flagellin H9 | 69,000 |
| Heat shock protein 70 | 70,000 |
| Calpain-1 (human erythrocytes) | 112,00 |
| C reactive Protein (5 × 25 kDa) | 115,000 |
| Myeloperoxidase (neutrophils) | 150,000 |
| NOS synthase | 150,000 |

The following examples are intended to be exemplary and non-limiting.

### Example 1: Base Sorbent Synthesis CY14175 & CY15077

Reactor Setup: a 4-neck glass lid was affixed to a 3 L jacketed cylindrical glass reaction vessel using a stainless steel flange clamp and PFTE gasket. The lid was fitted with a PFTE stirrer bearing, RTD adapter, and water-cooled reflux condenser. A stainless steel stirring shaft having five 60° agitators was fit through the stirrer bearing and inserted into a digital overhead stirrer. An RTD was fit through the corresponding adapter, and connected to a PolyStat circulating heating and chilling unit. Compatible tubing was used to connect the inlet and outlet of the reaction vessel jacket to the appropriate ports on the PolyStat. The unused port in the lid was used for charging the reactor and was plugged at all other times.

Polymerization: Aqueous phase and organic phase compositions are shown below, in Table I and Table II, respectively. Ultrapure water was split into approximately equal parts in two separate Erlenmeyer flasks, each containing a PFTE coated magnetic stir bar. Poly(vinyl alcohol) (PVA), having a degree of hydrolysis of 85.0 to 89.0 mol percent and a viscosity of 23.0 to 27.0 cP in a 4% aqueous solution at 20° C., was dispersed into the water in the first flask and heated to 80° C. on a hot plate with agitation. Salts (see Table 1, MSP, DSP, TSP and Sodium nitrite) were dispersed into the water in the second flask and heated to 80° C. on a hot plate with agitation. Circulation of heat transfer fluid from the PolyStat through the reaction vessel jacket was started, and fluid temperature heated to 60° C. Once PVA and salts dissolved, both solutions were charged to the reactor, one at a time, using a glass funnel. The digital overhead stirrer was powered on and the rpm set to a value to form appropriate droplet sizes upon organic phase addition. Temperature of the aqueous phase in the kettle was set to 70° C. The organic phase was prepared by adding benzoyl peroxide (BPO) to the divinylbenzene (DVB) in a 2 L Erlenmeyer flask and swirling until completely dissolved. 2,2,4-trimethylpentane and toluene were added to the flask, which was swirled to mix well. Once the temperature of the aqueous phase in the reactor reached 70° C., the organic phase was charged into the reactor using a narrow-necked glass funnel. Temperature of the reaction volume dropped upon the organic addition. A temperature program for the PolyStat was started, heating the reaction volume from to 77° C. over 30 minutes, 77 to 80° C. over 30 minutes, holding the temperature at 80° C. for 960 minutes, and cooling to 20° C. over 60 minutes.

TABLE I
Aqueous Phase Composition

| Reagent | Mass (g) |
|---|---|
| Ultrapure water | 1500.000 |
| Poly(vinyl alcohol) (PVA) | 4.448 |
| Monosodium phosphate (MSP) | 4.602 |
| Disodium phosphate (DSP) | 15.339 |
| Trisodium phosphate (TSP) | 9.510 |
| Sodium nitrite | 0.046 |
| Total | 1533.899 |

TABLE II
Organic Phase Compositions

| | CY14175 | CY15077 |
|---|---|---|
| Reagent | Mass (g) | Mass (g) |
| Divinylbenzene, 63% (DVB) | 508.751 | 498.383 |
| 2,2,4-trimethylpentane (Isooctane) | 384.815 | 482.745 |
| Toluene | 335.004 | 222.404 |
| Benzoyl peroxide, 98% (BPO) | 3.816 | 3.738 |
| Total (excluding BPO) | 1228.571 | 1203.532 |

Work-up: reaction volume level in the reactor was marked. Overhead stirrer agitation was stopped, residual liquid siphoned out of the reactor, and the reactor filled to the mark with ultrapure water at room temperature. Overhead stirrer agitation was restarted and the slurry heated to 70° C. as quickly as possible. After 30 minutes, agitation was stopped and residual liquid siphoned out. Polymer beads were washed five times in this manner. During the final wash, the slurry temperature was cooled to room temperature. After the final water wash, polymer beads were washed with 99% isopropyl alcohol (IPA) in the same manner. 99% IPA was siphoned out and replaced with 70% IPA before transferring the slurry into a clean 4 L glass container. Unless noted otherwise, on an as-needed basis the polymer was steam stripped in a stainless steel tube for 8 hours, rewet in 70% IPA, transferred into DI water, sieved to obtain only the portion of beads having diameters between 300 and 60011m, and dried at 100° C. until no further weight loss on drying was observed.

Cumulative pore volume data for polymers CY14175 and CY15077, measured by nitrogen desorption isotherm and mercury intrusion porosimetry, respectively, are shown below in Tables III and IV, respectively.

TABLE III
Nitrogen Desorption Isotherm Data for CY14175

| Pore Diameter | Pore size | Cumulative Pore |
|---|---|---|
| Range (Å) | Diameter (Å) | Volume (cm³/g) |
| 1411.9-1126.5 | 1236.809577 | 0.018062878 |
| 1126.5-981.7 | 1043.979923 | 0.038442381 |
| 981.7-752.9 | 836.7828769 | 0.141559621 |
| 752.9-659.9 | 700.1024343 | 0.24336622 |
| 659.9-572.0 | 609.4657394 | 0.416511969 |
| 572.0-483.1 | 519.8089977 | 0.646318614 |
| 483.1-449.8 | 465.2234212 | 0.730406771 |
| 449.8-401.4 | 422.7246485 | 0.849167577 |
| 401.4-354.1 | 374.6289335 | 0.956165766 |
| 354.1-337.9 | 345.6019761 | 0.997336398 |
| 337.9-313.5 | 324.758962 | 1.0547802 |
| 313.5-290.8 | 301.2432086 | 1.09667858 |
| 290.8-262.8 | 275.299967 | 1.164042391 |
| 262.8-247.2 | 254.510376 | 1.199751164 |
| 247.2-233.6 | 240.0176376 | 1.228796957 |
| 233.6-220.1 | 226.435352 | 1.256631669 |
| 220.1-208.6 | 213.9982044 | 1.283063762 |
| 208.6-130.5 | 151.2300725 | 1.464027373 |
| 130.5-105.7 | 115.2567614 | 1.527062065 |
| 105.7-82.8 | 91.14860242 | 1.592486039 |
| 82.8-67.6 | 73.42901881 | 1.641003444 |
| 67.6-57.5 | 61.59836256 | 1.6763711 |
| 57.5-51.6 | 54.15491457 | 1.699539142 |
| 51.6-45.0 | 47.72291376 | 1.728282889 |
| 45.0-39.8 | 42.01726183 | 1.752728216 |
| 39.8-35.8 | 37.55877213 | 1.779016164 |
| 35.8-31.8 | 33.51596841 | 1.8086605 |
| 31.8-28.7 | 30.02327371 | 1.82963357 |
| 28.7-26.0 | 27.18773181 | 1.850084632 |
| 26.0-23.3 | 24.46989555 | 1.87529426 |
| 23.3-20.9 | 21.92055755 | 1.902736527 |
| 20.9-18.5 | 19.52461159 | 1.935789448 |
| 18.5-16.2 | 17.16324429 | 1.97779901 |

TABLE IV
Mercury Intrusion Data for CY15077

| Pore size | Cumulative |
|---|---|
| Diameter (Å) | Intrusion (mL/g) |
| 226299.0625 | 3.40136E−30 |
| 213166.0781 | 0.001678752 |
| 201295.1563 | 0.002518128 |
| 172635.8125 | 0.004364755 |
| 139538.0625 | 0.007554384 |
| 113120.7813 | 0.011919139 |
| 90542.36719 | 0.01645177 |
| 78733.25781 | 0.0203129 |
| 72446.375 | 0.022327403 |
| 60340.40234 | 0.027867284 |
| 48343.83984 | 0.035327822 |
| 39009.13672 | 0.040918175 |
| 32136.4082 | 0.04899035 |
| 25330.65625 | 0.063195683 |
| 20981.51563 | 0.079529688 |
| 16219.86426 | 0.108860672 |
| 13252.41211 | 0.141730919 |
| 10501.53613 | 0.193969816 |
| 8359.911133 | 0.262399256 |
| 6786.30127 | 0.345866203 |
| 5538.122559 | 0.438174427 |
| 4337.931152 | 0.563276172 |
| 3501.674805 | 0.681870878 |
| 2838.742188 | 0.804727197 |
| 2593.016846 | 0.865813017 |
| 2266.688965 | 0.938610673 |
| 1831.041748 | 1.056586146 |
| 1509.850708 | 1.163395643 |
| 1394.006104 | 1.21002543 |
| 1294.780151 | 1.257248282 |
| 1207.692627 | 1.293158531 |
| 1131.860962 | 1.326992273 |
| 1065.099976 | 1.35812819 |
| 953.1816406 | 1.405935764 |
| 884.0358887 | 1.445426106 |
| 823.5491333 | 1.478719592 |
| 770.9108276 | 1.510579824 |
| 722.4724731 | 1.537048101 |
| 684.6119995 | 1.564400196 |
| 672.187561 | 1.581117511 |
| 636.7885742 | 1.60271585 |
| 604.7248535 | 1.621845484 |
| 558.1287231 | 1.651492 |
| 518.2624512 | 1.678913713 |
| 483.5536499 | 1.708594561 |
| 453.5110779 | 1.735918999 |
| 426.9998474 | 1.755934 |
| 403.1251526 | 1.783603072 |
| 382.7776794 | 1.793849826 |
| 362.7162476 | 1.817784309 |
| 342.3734436 | 1.838774562 |
| 330.1105042 | 1.851493955 |
| 315.5238037 | 1.869742155 |
| 302.2973938 | 1.885128617 |
| 290.2946777 | 1.895119786 |
| 279.1246643 | 1.912378907 |
| 268.7442627 | 1.924305081 |
| 259.1106873 | 1.936048627 |
| 241.8737793 | 1.955100656 |
| 226.7678223 | 1.972970247 |
| 213.3626251 | 1.988123298 |
| 201.4908142 | 2.007521152 |
| 194.9888611 | 2.022114754 |
| 188.9506989 | 2.033871174 |
| 180.582901 | 2.035052776 |
| 172.8530121 | 2.050720692 |
| 164.9621735 | 2.062945843 |
| 157.8110657 | 2.071056128 |
| 151.1540375 | 2.082133055 |
| 143.9185333 | 2.096480608 |
| 138.4670563 | 2.106938839 |
| 132.8492737 | 2.119287968 |
| 129.5760345 | 2.126605988 |
| 126.5438614 | 2.126605988 |
| 124.2635574 | 2.132267475 |
| 120.8976135 | 2.141504765 |
| 117.3792267 | 2.150759459 |
| 114.791893 | 2.154810667 |
| 111.9475937 | 2.162935257 |
| 108.8830032 | 2.167646885 |
| 106.6480179 | 2.174062729 |
| 104.5217743 | 2.179908991 |
| 102.4295197 | 2.179908991 |
| 100.1580353 | 2.182951927 |
| 98.29322052 | 2.184018135 |
| 96.44822693 | 2.191127539 |
| 94.42159271 | 2.198545218 |
| 91.52587891 | 2.209161043 |
| 89.25807953 | 2.209312439 |
| 87.0777359 | 2.215425491 |
| 85.42358398 | 2.221472025 |
| 83.62612915 | 2.232139587 |
| 82.11174011 | 2.237514496 |
| 79.91614532 | 2.239231586 |
| 78.01462555 | 2.239560127 |
| 76.19993591 | 2.239560127 |
| 75.09249115 | 2.239560127 |
| 73.41201019 | 2.239560127 |
| 72.23709869 | 2.240245819 |
| 71.09960175 | 2.242422104 |
| 69.86301422 | 2.243849993 |
| 68.40761566 | 2.257676363 |
| 67.13697815 | 2.259181261 |
| 66.03359222 | 2.266284466 |
| 65.08189392 | 2.270181179 |
| 64.04368591 | 2.272682428 |
| 62.38490295 | 2.280714512 |
| 61.32764053 | 2.280714512 |
| 60.30379868 | 2.287917852 |
| 59.41370392 | 2.287917852 |
| 58.54679489 | 2.293802738 |
| 57.79866409 | 2.297607183 |
| 56.88977814 | 2.299046278 |
| 55.9213295 | 2.302111387 |
| 54.98665237 | 2.303381443 |

### Example 2: Polymer Modification CY15065

250 mL base polymer CY14175, wetted in DI water, was added to a 500 mL jacketed glass reactor which was equipped with a Teflon coated agitator and RTD probe. 90 mL excess DI water was added to the reactor, and the slurry mixed at 90 RPM. Reaction temperature was set to 20° C. Three separate additions were prepared; 1.4 g ammonium persulfate in 14 mL DI water, 0.7 g N-vinylpyrrolidinone in 21 mL DI water, and 1.5 g N,N,N,N-tetramethylethylenediamine in 7 mL DI water. Reaction temperature setpoint was increased to and monitored closely. Ammonium persulfate solution was added once reaction temperature reached 30° C. N,N,N,N-tetramethylethylenediamine solution was added once reaction temperature reached 35° C. N-vinylpyrrolidinone solution was added once reaction temperature reached 39° C. Reaction was then maintained at 40° C. for 2 hours, before decreasing temperature to 25° C.

Work-up: reaction volume level in the reactor was marked. Overhead stirrer agitation was stopped, residual liquid siphoned out of the reactor, and the reactor filled to the mark with ultrapure water at room temperature. Overhead stirrer agitation was restarted. After minutes, agitation was stopped and residual liquid siphoned out. Polymer beads were washed three times in this manner. The polymer was steam stripped in a stainless steel tube for 8 hours, rewet in 70% IPA, then transferred into DI water.

Cumulative pore volume data for polymers CY15065, measured by nitrogen desorption isotherm, is shown below in Table V.

TABLE V
Nitrogen Desorption Isotherm Data for CY15065

| Pore Diameter | Pore size | Cumulative Pore |
|---|---|---|
| Range (Å) | Diameter (Å) | Volume (cm³/g) |
| 3735.2-1226.3 | 1462.700326 | 0.005546248 |
| 1226.3-724.9 | 851.9599269 | 0.012549654 |
| 724.9-656.2 | 686.9797148 | 0.01499268 |
| 656.2-595.3 | 622.6438437 | 0.020898533 |
| 595.3-566.7 | 580.2249215 | 0.033282221 |
| 566.7-511.2 | 535.9524733 | 0.111667343 |
| 511.2-459.4 | 482.4242892 | 0.244617735 |
| 459.4-410.9 | 432.3534717 | 0.366033907 |
| 410.9-376.5 | 392.140848 | 0.468167046 |
| 376.5-334.5 | 352.8905481 | 0.63111569 |
| 334.5-298.7 | 314.4631626 | 0.753980937 |
| 298.7-291.1 | 294.7961544 | 0.786627632 |
| 291.1-273.8 | 281.8886994 | 0.844237668 |
| 273.8-256.6 | 264.6128479 | 0.91291745 |
| 256.6-228.9 | 241.1063341 | 0.990966112 |
| 228.9-224.9 | 226.9020278 | 1.007532081 |
| 224.9-209.7 | 216.7455697 | 1.046516069 |
| 209.7-141.7 | 162.0655488 | 1.225860688 |
| 141.7-103.9 | 116.5968304 | 1.329424083 |
| 103.9-82.9 | 90.79492123 | 1.388864147 |
| 82.9-69.3 | 74.73718587 | 1.431186463 |
| 69.3-59.3 | 63.39311832 | 1.465462484 |
| 59.3-50.9 | 54.36935206 | 1.495795233 |
| 50.9-44.8 | 47.39226606 | 1.521105084 |
| 44.8-39.5 | 41.71847385 | 1.544891234 |
| 39.5-35.3 | 37.12325172 | 1.568519104 |
| 35.3-31.4 | 33.1071979 | 1.594220982 |
| 31.4-28.2 | 29.60557504 | 1.614149255 |
| 28.2-25.6 | 26.73665706 | 1.633426358 |
| 25.6-22.8 | 24.01512381 | 1.656221583 |
| 22.8-20.5 | 21.49286805 | 1.680044553 |
| 20.5-18.2 | 19.15056378 | 1.707534945 |

### Example 3: Removal from Whole Bovine Blood in a Recirculation Model

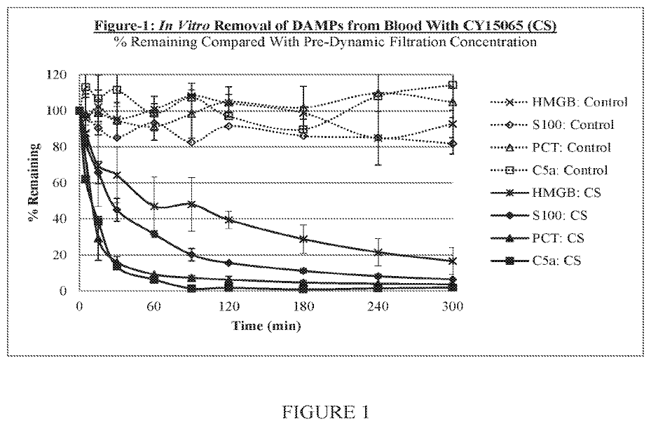

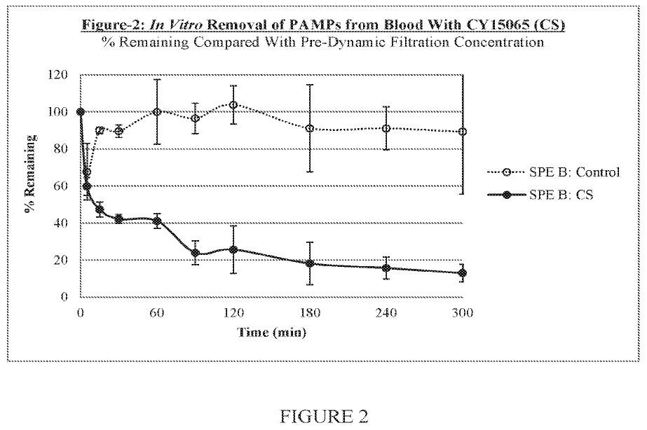

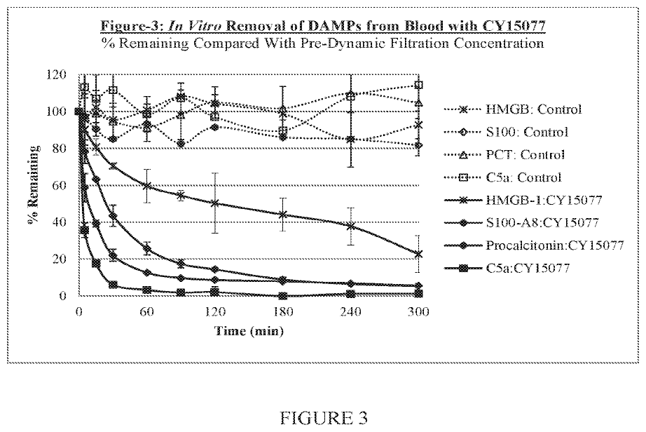

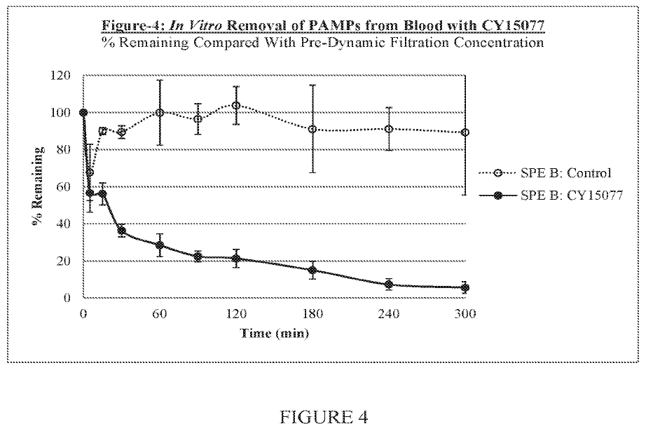

Purified proteins were added to 300 mL 3.8% citrated whole bovine blood (Lampire Biologicals) at expected clinical concentrations and recirculated through a 20 mL polymer-filled device or control (no bead) device at a flow rate of 140 mL/min for five hours. Proteins and initial concentrations were: S100A8 at 50 ng/mL, complement C5a at 25 ng/mL, procalcitonin at 16 ng/mL, HMGB-1 at 100 ng/mL, and SPE B at 100 ng/mL. Plasma was analyzed by enzyme-linked immunosorbent assay (ELISA) following manufacturer instructions (S100, and C5a, duosets (R&D Systems); procalcitonin (Sigma), HMGB-1 (Chondrex ELISA); and toxins (Toxin Technologies). Removal data from experiments using polymer CY15065 are shown below in FIG. 1 and FIG. 2 for DAMPs and PAMPs, respectively. For brevity, CY15065 is denoted ‘CS’ in the figure legends. Removal data from experiments using polymer CY15077 are shown below in FIG. 3 and FIG. 4 for DAMPs and PAMPs, respectively.

1-14. (canceled)

15. A method of removing (i) pathogen-associated molecular pattern molecules (PAMPS) and (ii) damage-associated molecular pattern molecules (DAMPS) from a physiological fluid comprising:
contacting the physiological fluid with a polymer comprising a plurality of pores in a range of from 50 Å to 40,000 Å and having a total volume of pore sizes in a range of from about cc/g to about 5.0 cc/g dry polymer; and
wherein the PAMPS and DAMPS have a molecular weight of from about 0.5 kDa to about 1,000 kDa.

16. The method of claim 15, wherein the polymer is hemocompatible.

17. The method of claim 15, wherein the polymer has a geometry that is a spherical bead.

18. The method of claim 15, wherein the PAMPs and DAMPS comprise one or more of flagellins, lipopeptides, formyl peptides, mycotoxins, exotoxins, endotoxins, lipoteichoic acid, cytolysins, superantigens, proteases, lipases, amylases, enzymes, peptides including bradykinin, activated complement, soluble receptors, soluble CD40 ligand, bioactive lipids, oxidized lipids, cellular DNA, mitochondrial DNA, pathogen or host derived RNA, cell-free hemoglobin, cell-free myoglobin, growth factors, peptidoglycans, glycoproteins, released intracellular components, cell wall or viral envelope components, Polyinosinic:polycytidylic acid (poly I:C), prions, toxins, bacterial and viral toxins, drugs, vasoactive substances, and foreign antigens.

19. The method of claim 15, wherein the polymer is made using suspension polymerization.

20. The method of claim 15, wherein the polymer is a hypercrosslinked polymer.

21. The method of claim 17, wherein the spherical bead has a biocompatible hydrogel coating.

22. The method of claim 15, wherein the polymer is formed and subsequently modified to be biocompatible.

23. The method of claim 15, wherein the polymer is housed in a container suitable to retain the polymer and for transfusion of a physiological fluid selected from whole blood, packed red blood cells, platelets, albumin, plasma and combinations thereof.

24. The method of claim 15, wherein the polymer is in a device suitable to retain the polymer and be incorporated into an extracorporeal circuit.

25. The method of claim 15, wherein free polymer (i.e. not contained) is used to treat the physiologic fluids.

26. The method of claim 15, wherein physiological fluid is selected from whole blood, packed red blood cells, platelets, albumin, plasma and combinations thereof.

27. A method of perfusion comprising:
passing a physiologic fluid once through or by way of a suitable extracorporeal circuit through a device once or many times comprising a polymer comprising a plurality of pores in a range of from 50 Å to 40,000 Å and having a total volume of pore sizes in a range of from about cc/g to about 5.0 cc/g dry polymer; and
removing (i) pathogen-associated molecular pattern molecules (PAMPS) and (ii) damage-associated molecular pattern molecules (DAMPS) from the physiological fluid;
wherein the PAMPS and DAMPS have a molecular weight of from about 0.5 kDa to about 1,000 kDa.

28. The method of claim 27, wherein the polymer is hemocompatible.

29. The method of claim 27, wherein the polymer has a geometry that is a spherical bead.

30. The method of claim 27, wherein the PAMPs and DAMPS comprise one or more of flagellins, lipopeptides, formyl peptides, mycotoxins, exotoxins, endotoxins, lipoteichoic acid, cytolysins, superantigens, proteases, lipases, amylases, enzymes, peptides including bradykinin, activated complement, soluble receptors, soluble CD40 ligand, bioactive lipids, oxidized lipids, cellular DNA, mitochondrial DNA, pathogen or host derived RNA, cell-free hemoglobin, cell-free myoglobin, growth factors, peptidoglycans, glycoproteins, released intracellular components, cell wall or viral envelope components, Polyinosinic:polycytidylic acid (poly I:C), prions, toxins, bacterial and viral toxins, drugs, vasoactive substances, and foreign antigens.

31. The method of claim 27, wherein the polymer is made using suspension polymerization.

32. The method of claim 27, wherein the polymer is a hypercrosslinked polymer.

33. The method of claim 29, wherein the spherical bead has a biocompatible hydrogel coating.

34. The method of claim 27, wherein the polymer is formed and subsequently modified to be biocompatible.

35. The method of claim 27, wherein physiological fluid is selected from whole blood, packed red blood cells, platelets, albumin, plasma and combinations thereof.

36. A method for treating contamination with pathogen-associated molecular pattern molecules (PAMPS) and/or damage-associated molecular pattern molecules (DAMPS), comprising
contacting physiological fluid of a patient with a polymer comprising a plurality of pores in a range of from 50 Å to 40,000 Å and having a total volume of pore sizes in a range of from about 0.5 cc/g to about 5.0 cc/g dry polymer;
wherein the PAMPS and DAMPS have a molecular weight of from about 0.5 kDa to about 1,000 kDa.

37. The method of claim 36, wherein the polymer is hemocompatible.

38. The method of claim 36, wherein the polymer has a geometry that is a spherical bead.

39. The method of claim 36, wherein the PAMPs and DAMPS comprise one or more of flagellins, lipopeptides, formyl peptides, mycotoxins, exotoxins, endotoxins, lipoteichoic acid, cytolysins, superantigens, proteases, lipases, amylases, enzymes, peptides including bradykinin, activated complement, soluble receptors, soluble CD40 ligand, bioactive lipids, oxidized lipids, cellular DNA, mitochondrial DNA, pathogen or host derived RNA, cell-free hemoglobin, cell-free myoglobin, growth factors, peptidoglycans, glycoproteins, released intracellular components, cell wall or viral envelope components, Polyinosinic:polycytidylic acid (poly I:C), prions, toxins, bacterial and viral toxins, drugs, vasoactive substances, and foreign antigens.

40. The method of claim 36, wherein the polymer is made using suspension polymerization.

41. The method of claim 36, wherein the polymer is a hypercrosslinked polymer.

42. The method of claim 38, wherein the spherical bead has a biocompatible hydrogel coating.

43. The method of claim 36, wherein the polymer is formed and subsequently modified to be biocompatible.

44. The method of claim 36, wherein the polymer is housed in a container suitable to retain the polymer and for transfusion of a physiological fluid selected from whole blood, packed red blood cells, platelets, albumin, plasma and combinations thereof.

45. The method of claim 36, wherein the polymer is in a device suitable to retain the polymer and be incorporated into an extracorporeal circuit.

46. The method of claim 36, wherein free polymer (i.e. not contained) is used to treat the physiologic fluids.

47. The method of claim 36, wherein physiological fluid is selected from whole blood, packed red blood cells, platelets, albumin, plasma and combinations thereof.
